(19) United States
(12) Patent Application Publication
(10) Pub. No.: US 2025/0345579 A1
(43) Pub. Date: Nov. 13, 2025

(54) Biological Tissue Transdermal Patch

(71) Applicant: Nippon Telegraph and Telephone Corporation, Tokyo (JP)

(72) Inventors: Masaya Nohara, Musashino-shi, Tokyo (JP); Hiroaki Taguchi, Musashino-shi, Tokyo (JP); Sho Okubo, Musashino-shi, Tokyo (JP); Shuhei Sakamoto, Musashino-shi, Tokyo (JP); Takeshi Komatsu, Musashino-shi, Tokyo (JP)

(21) Appl. No.: 18/869,482

(22) PCT Filed: May 26, 2022
(86) PCT No.: PCT/JP2022/021622

§ 371 Date: Nov. 26, 2024

(51) Int. Cl. A61M 37/00; A61N 1/30

(52) U.S. Cl. CPC A61M 37/0015; A61N 1/30; A61M 2037/0023

(57) ABSTRACT

A patch for sticking to a biological tissue includes a battery unit, and a soluble microneedle in contact with the battery unit, in which the soluble microneedle contains an active ingredient, and a battery reaction is started by inserting the soluble microneedle into the biological tissue.

### TECHNICAL FIELD

The present invention relates to a patch for sticking to a biological tissue that is used by being stuck to a biological tissue.

### BACKGROUND ART

Liquid and cream-based cosmetics and pharmaceuticals are widely prevalent. A method for allowing active ingredients of the cosmetics and pharmaceuticals to permeate into a living body by a weak current has attracted attention. A technique using a weak current is known to be expected to have an effect of enhancing cell activation and drug permeation, but an expensive and large power supply device is required.

In order to solve such a problem, a patch for sticking to a biological tissue including a power supply device using a general dry battery is known. However, since the power supply device using a general dry battery uses a material, a rare metal, or the like, which is harmful to the dry battery and the power supply device, there are problems such as a reduction in an environmental load and simplification of disposal.

A patch for sticking to a biological tissue with a low environmental load has also been studied (Patent Literature 1 and Non Patent Literature 1).

### CITATION LIST

### Patent Literature

Patent Literature 1: WO2018/194079 A1

### Non Patent Literature

Non Patent Literature 1: Yudai Ogawa, Koichiro Kato, Takeo Miyake, Kuniaki Nagamine, Takuya Ofuji, Syuhei Yoshino, and Matsuhiko Nishizawa, “Organic Transdermal Iontophoresis Patch with Built-in Biofuel Cell”, Advanced Healthcare Materials, WILEY-VCH Verlag GmbH & Co. KGaA, Weinheim, 2015, Volume 4, Issue 4, pp. 506-510

### SUMMARY OF INVENTION

### Technical Problem

The patch for sticking to a biological tissue in Patent Literature 1 uses the principle of a metal-air battery, and the patch for sticking to a biological tissue in Non Patent Literature 1 uses the principle of a biofuel cell.

A biological tissue has a skin barrier function, and in general, an active ingredient that permeates into the biological tissue is said to have a molecular weight of 500 daltons or less (500 dalton rule). The patch for sticking to a biological tissue of Patent Literature 1 and Non Patent Literature 1 has an effect of easily promoting the permeation of the active ingredient, but there is a problem of promoting the permeation of a high-molecular-weight active ingredient having a molecular weight of 500 or more.

Further, in the metal-air battery and the biofuel cell described above, there is also a problem that an electrode corrodes as a liquid, creamy, or gel-shaped active ingredient is continuously in contact with the battery. For this reason, there is a step of removing a partition wall separating the active ingredient from the battery before using the patch for sticking to a biological tissue, and a complicated step exists as a burden on a user.

The present invention has been made in view of the above, and an object of the present invention is to provide a patch for sticking to a biological tissue, which promotes the permeation of a high-molecular-weight active ingredient and is capable of easily starting a battery reaction.

### Solution to Problem

In order to solve the above problems, a patch for sticking to a biological tissue according to one aspect of the present invention is a patch for sticking to a biological tissue that is used by being stuck to a biological tissue, the patch for sticking to a biological tissue, including: a battery unit; and a soluble microneedle in contact with the battery unit, in which the soluble microneedle contains an active ingredient, and a battery reaction is started by inserting the soluble microneedle into the biological tissue.

### Advantageous Effects of Invention

According to the present invention, it is possible to provide a patch for sticking to a biological tissue, which promotes the permeation of the high-molecular-weight active ingredient and is capable of easily starting the battery reaction.

### BRIEF DESCRIPTION OF DRAWINGS

FIG. 1 is a plan view of a patch for sticking to a biological tissue according to this embodiment.

FIG. 2 is a side view of the patch for sticking to a biological tissue in FIG. 1.

FIG. 3 is a diagram illustrating a state in which the patch for sticking to a biological tissue in FIG. 1 is used by being stuck to a biological tissue.

FIG. 4 is a diagram schematically illustrating a configuration of a patch for sticking to a biological tissue separated into a soluble microneedle in contact with a positive electrode part and a soluble microneedle in contact with a negative electrode part.

FIG. 5 is a flowchart illustrating a method for producing bacteria-produced cellulose carbide.

FIG. 6 is a flowchart illustrating a step of supporting a catalyst on the bacteria-produced cellulose carbide.

FIG. 7 is a flowchart illustrating another method for producing a positive electrode.

FIG. 8 is a flowchart illustrating a method for producing a negative electrode.

FIG. 9 is an exploded perspective view of a patch for sticking to a biological tissue of Example 1.

FIG. 10 is a sectional view of the patch for sticking to a biological tissue of Example 1.

FIG. 11 is a diagram illustrating a configuration of a test device.

FIG. 12 is a diagram illustrating a state in which the patch for sticking to a biological tissue is disposed in the test device.

FIG. 13 is a perspective view of a patch for sticking to a biological tissue of Comparative Example 1.

FIG. 14 is a sectional view of the patch for sticking to a biological tissue of Comparative Example 1.

FIG. 15 is a graph illustrating a measurement result.

FIG. 16 is an exploded perspective view of a patch for sticking to a biological tissue of Example 2.

FIG. 17 is a sectional view of the patch for sticking to a biological tissue of Example 2.

FIG. 18 is an exploded perspective view of a patch for sticking to a biological tissue of Comparative Example 3.

FIG. 19 is a sectional view of the patch for sticking to a biological tissue of Comparative Example 3.

### DESCRIPTION OF EMBODIMENTS

Hereinafter, an embodiment of the present invention will be described with reference to the drawings.

### (Configuration of Patch for Sticking to Biological Tissue)

A patch for sticking to a biological tissue of this embodiment is a patch for allowing an active ingredient to permeate into a biological tissue by electricity generated by a reaction similar to that in a general magnesium-air battery. Specifically, the patch for sticking to a biological tissue of this embodiment is attached to the biological tissue to allow the active ingredient to permeate into the biological tissue with a weak current.

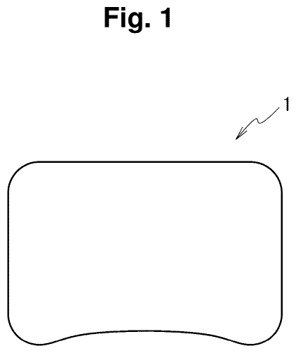

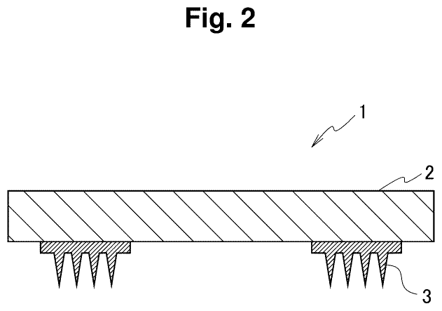

FIG. 1 is a plan view illustrating the configuration of a patch for sticking to a biological tissue according to this embodiment, and FIG. 2 is a side view illustrating the configuration of the patch for sticking to a biological tissue according to this embodiment. A patch 1 for sticking to a biological tissue illustrated in FIG. 2 includes a battery unit 2, and a soluble microneedle 3 in contact with the battery unit 2. The battery unit 2 does not include an electrolyte that is required for a general battery, and is stored in a state where no battery reaction occurs. The patch 1 for sticking to a biological tissue is used by being attached to a biological tissue. The patch 1 for sticking to a biological tissue starts a battery reaction by inserting the soluble microneedle 3 into the biological tissue.

The soluble microneedle 3 contains an active ingredient. As a base material of the soluble microneedle 3, a material that can be used for a microneedle of the related art can be basically used insofar as the material is dissolved in the biological tissue after being inserted into the biological tissue. The soluble microneedle 3 is preferably a thermoplastic polymer from the viewpoint of mass producibility, and is more preferably a material ensuring biological safety.

In addition to the above configuration, the patch 1 for sticking to a biological tissue can include a structural member such as an exterior film, a case, an adhesive, and a metal foil, and an element required for a general magnesium-air battery. As these, conventionally known ones can be used.

When the patch 1 for sticking to a biological tissue is used, the soluble microneedle 3 is inserted into a biological tissue 100. When the soluble microneedle 3 starts to be dissolved in the biological tissue 100 due to moisture in the biological tissue 100, the soluble microneedle 3 containing the active ingredient functions similarly to the electrolyte, and the battery reaction is started in the battery unit 2.

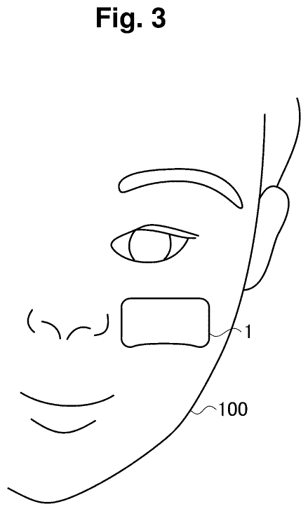

For example, as illustrated in FIG. 3, the soluble microneedle 3 is inserted into the biological tissue 100. As a method for inserting the soluble microneedle 3 into the biological tissue 100, a method for pressing the patch 1 for sticking to a biological tissue with a finger to apply a pressure is suitable because the method is easy and low cost. The method for inserting the soluble microneedle 3 into the biological tissue 100 is not particularly limited, and for example, when the pressure is insufficient and the soluble microneedle 3 is not capable of being inserted, an insertion jig or the like may be used.

After the battery reaction is started, the patch 1 for sticking to a biological tissue may be continuously in contact with the biological tissue 100 until the active ingredient contained in the soluble microneedle 3 is sufficiently diffused into the biological tissue 100. The shapes of the patch 1 for sticking to a biological tissue and the battery unit 2 are not particularly limited. For example, a patch shape, a face mask shape, an eye mask shape, a glove shape, a bandage shape, an adhesive plaster shape, or a poultice shape may be used.

### (Configuration of Battery Unit)

Next, the configuration of the battery unit 2 will be described.

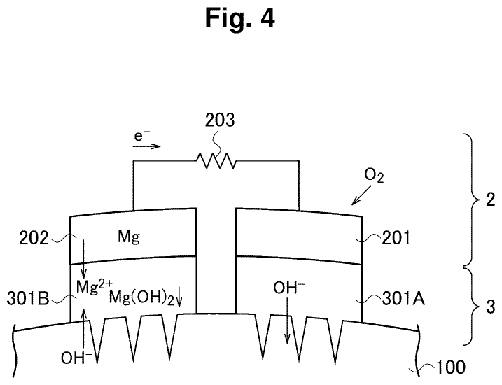

FIG. 4 is a diagram schematically illustrating an example of the configuration of the battery unit 2 and the soluble microneedle 3.

The illustrated battery unit 2 includes a positive electrode 201, a negative electrode 202 containing magnesium, and a conductive layer 203 electrically connected to the positive electrode 201 and the negative electrode 202. Unlike a general magnesium-air battery, the battery unit 2 of this embodiment does not include an electrolyte. The battery unit 2 is used in contact with the soluble microneedle 3. The illustrated soluble microneedle 3: includes a positive electrode part soluble microneedle 301A and a negative electrode part soluble microneedle 301B.

Here, an electrode reaction in the positive electrode 201 and the negative electrode 202 will be described.

On the surface of the positive electrode 201, water absorbed by the soluble microneedle from the biological tissue and oxygen in the air are in contact with each other, and thus, a reaction represented by the following formula (1) proceeds.

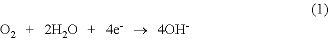

On the other hand, in the negative electrode 202 in contact with the dissolved soluble microneedle, a reaction represented by the following formula (2) proceeds. Specifically, magnesium configuring the negative electrode 202 emits electrons and is dissolved as magnesium ions in the dissolved soluble microneedle and the active ingredient.

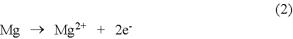

Such reactions are performed through the biological tissue 100. In the patch 1 for sticking to a biological tissue of FIG. 4, the positive electrode part soluble microneedle 301A and the active ingredient are introduced into the biological tissue 100 together with hydroxide ions (OH⁻).

The entire reaction of the battery reaction is represented by the following formula (3), which is a reaction for producing magnesium hydroxide.

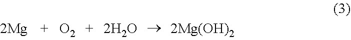

The theoretical electromotive force is approximately 2.7 V. FIG. 4 illustrates a compound relevant to the reaction, together with the constituent of the patch 1 for sticking to a biological tissue.

Hereinafter, each constituent of the battery unit 2 will be described.

### (I) Positive Electrode

As the positive electrode 201, a positive electrode used in a general magnesium-air battery can be used. For example, carbon, a metal, an oxide, a nitride, a carbide, a sulfide, and a phosphide can be used. Two or more types thereof may be mixed. The positive electrode 201 can be produced by a known process of molding a carbon powder with a binder. Since a resin containing fluorine is generally used as the binder, a hydrofluoric acid is generated when the positive electrode 201 is burned by disposal or the like. Therefore, there is room for improvement such as safety improvement and a reduction in an environmental load.

The positive electrode 201 of this embodiment may contain cellulose carbide having a three-dimensional network structure. Specifically, in the positive electrode 201, bacteria-produced cellulose carbide or cellulose nanofiber carbon is used for the positive electrode 201, and thus, a resin containing fluorine is not used. The bacteria-produced cellulose carbide that is used for the positive electrode 201 has a three-dimensional network structure of bacteria-produced carbonized cellulose, and for example, the average pore size is preferably 0.1 to 50 μm, and more preferably 0.1 to 2 μm. The average pore size is a value measured by a mercury intrusion technique. The cellulose nanofiber carbon that is used for the positive electrode 201 has a three-dimensional network structure of a carbonized cellulose nanofiber, and for example, the fiber diameter is preferably 5 to 500 nm, and preferably 20 to 200 nm.

The positive electrode 201 may support a catalyst. Examples of the catalyst include a metal, an oxide, a nitride, a carbide, a sulfide, and a phosphide. Two or more types thereof may be mixed. As the metal, iron, manganese, copper, nickel, silver, gold, platinum, cobalt, ruthenium, molybdenum, titanium, chromium, gallium, praseodymium, aluminum, silicon, and tin can be used. An alloy containing two or more types thereof may be used. The oxide is preferably an oxide containing one of the above metals or a composite oxide containing two or more of the above metals. In particular, iron oxide (Fe₂O₃) is preferable. It is preferable that the iron oxide exhibits particularly excellent catalytic performance and is not a rare metal. The metal oxide as the catalyst is preferably an amorphous metal oxide as a hydrate. For example, a hydrate of the transition metal oxide described above may be used. More specifically, an iron (III) oxide-n-hydrate may be used. Note that, n represents the number of moles of H₂O with respect to 1 mol of Fe₂O₃.

When nano-sized fine particles of an iron oxide hydrate (Fe₂O₃·nH₂O) are highly dispersed and attached (added) to the surface of the bacteria-produced cellulose carbide of the positive electrode 201, excellent performance can be exhibited. The content of the catalyst contained in the positive electrode 201 is 0.1 to 70 wt %, and is preferably 1 to 30 wt %, on the basis of the total weight of the positive electrode 201. As a transition metal oxide is added as the catalyst to the positive electrode 201, the performance of the battery unit 2 is greatly improved.

The reaction represented by the above formula (1) proceeds on the surface of the positive electrode 201. Therefore, it is important to generate a large amount of reaction sites inside the positive electrode 201, and it is desirable that the positive electrode 201 has a high specific surface area. For example, the specific surface area of the positive electrode 201 is preferably 200 m²/g or more, and more preferably 300 m²/g or more.

### (II) Negative Electrode

The negative electrode 202 contains a negative-electrode active material. The negative-electrode active material may be a material that can be used as a negative electrode material of the magnesium-air battery, that is, a material containing metallic magnesium and a magnesium-containing substance. The negative electrode 202, for example, may contain metallic magnesium, a sheet of metallic magnesium, or a magnesium powder.

The negative electrode 202 may contain at least one selected from the group consisting of magnesium, zinc, aluminum, iron, calcium, lithium, and sodium. That is, iron, zinc, aluminum, calcium, lithium, and sodium, which can be used as a metal-air battery other than magnesium, can also be used as the negative electrode material. Magnesium is most preferably used from the viewpoint of safety and battery output.

### (III) Conductive Layer

The conductive layer 203 is in contact with each of the positive electrode 201 and the negative electrode 202. The conductive layer 203 is not particularly limited insofar as the conductive layer is a material having conductivity. Examples thereof include a carbon cloth, a carbon sheet, a metal mesh, a metal wire, a conductive cloth, conductive rubber, and a conductive polymer. The rate of the battery reaction can be adjusted by adjusting the electric resistance value of the conductive layer 203. When the resistance value of the conductive layer 203 is increased, the rate of ion introduction of the active ingredient into the biological tissue 100 is slowed down. In a case where the ion introduction is excessively fast to cause pain, the resistance value of the conductive layer 203 may be increased. On the other hand, in a case where the ion introduction of the active ingredient into the biological tissue 100 is accelerated, the resistance value of the conductive layer 203 may be decreased.

### (Configuration of Soluble Microneedle)

In the example illustrated in FIG. 4, the illustrated soluble microneedle 3 includes the positive electrode part soluble microneedle 301A and the negative electrode part soluble microneedle 301B. That is, the soluble microneedle 3 is separated into the positive electrode part soluble microneedle 301A and the negative electrode part soluble microneedle 301B. The positive electrode part soluble microneedle 301A and the negative electrode part soluble microneedle 301B are not in contact with each other. Specifically, the positive electrode part soluble microneedle 301A is disposed to be in contact with the positive electrode 201 and not to be in contact with the negative electrode 202, the negative electrode part soluble microneedle 301B is disposed to be in contact with the negative electrode 202 and not to be in contact with the positive electrode 201, and the positive electrode part soluble microneedle 301A and the negative electrode part soluble microneedle 301B are used in the state of being inserted into the biological tissue 100.

In addition, as another configuration example of the soluble microneedle 3, the soluble microneedle 3 may not be separated into the positive electrode part and the negative electrode part.

The soluble microneedle may be a material that is capable of containing an active ingredient and does not have conductivity. Insofar as the soluble microneedle is dissolved in the biological tissue after being inserted into the biological tissue, basically, a material that can be used for a microneedle of the related art can be used, a thermoplastic polymer is preferable from the viewpoint of the possibility of mass production, and a material ensuring biological safety is more preferable.

Examples of a base material of the soluble microneedle include a polylactic acid, a poly(lactic-glycolic acid) copolymer, a polyglycolic acid, polyethylene terephthalate, nylon, polycarbonate, a cyclic olefin polymer (COP), and a mixture thereof, and more preferably a hyaluronic acid, dextran, polyvinyl pyrrolidone, sodium chondroitin sulfate, hydroxypropyl cellulose, polyvinyl alcohol, or a mixture thereof.

Specific examples of the active ingredient will be described below.

The needle length of the soluble microneedle is 0.2 mm to 1.0 mm, and is more preferably 0.4 mm to 1.0 mm. This is because, in the case of human skin, the thickness from the skin surface to the dermis layer where nerves, blood vessels, and lymphatic vessels are present is usually 0.1 mm to 0.2 mm, and thus, in a needle length of 0.2 mm or more, the active ingredient can be more effectively diffused into the biological tissue.

The needle density is preferably 20 to 400 needles/cm². The soluble microneedles stand on a substrate, and the density thereof may be uniform over the entire surface of the substrate, or may have a sparse and dense configuration. Further, there may be a region where the soluble microneedle is not present.

The soluble microneedle can be produced by using a mold. Press molding, injection molding, and the like can be performed, and the injection molding is desirable from the viewpoint of cost. In addition, a semiconductor manufacturing technology such as nanoimprinting and photoresist can also be applied.

In the case of the patch 1 for sticking to a biological tissue in which the soluble microneedle is stuck to the biological tissue, as illustrated in FIG. 4, it is preferable that the soluble microneedle is divided into the positive electrode part soluble microneedle 301A and the negative electrode part soluble microneedle 301B, which are not in contact with each other. This is because in a case where the positive electrode part soluble microneedle 301A and the negative electrode part soluble microneedle 301B are in contact with each other, the battery reaction proceeds without interposing the biological tissue, and the ion introduction effect of the active ingredient is weakened.

### (Active Ingredient)

Next, the active ingredient will be described.

The “active ingredient” of this embodiment refers to a chemical solution having an effect on a specific disease, a cosmetic solution for the purpose of cleaning and beautifying the human body, increasing the attractiveness, changing the appearance, and keeping the skin or hair healthy, water, alcohol, and the like.

The active ingredient may be a substance that is capable of transferring magnesium ions and hydroxide ions between the positive electrode 201 and the negative electrode 202 through the biological tissue 100 or the soluble microneedle 3.

Examples of the active ingredient include an aqueous solution containing organic and inorganic acids, a derivative thereof, and a salt thereof. Examples of the active ingredient include an amino acid ion, a chloride ion, a citrate ion, a lactate ion, a succinate ion, a phosphate ion, a malate ion, a pyrrolidone carboxylate ion, a sulfophenyl carbonate ion, a sulfate ion, a nitrate ion, a carbonate ion, and a perchlorate ion, as anion species. Examples of the amino acid include glycine, alanine, valine, leucine, isoleucine, phenylalanine, tyrosine, threonine, serine, proline, tryptophan, methionine, cysteine, an aspartic acid, a glutamic acid, asparagine, glutamine, lysine, arginine, histidine, hydroxyproline, cystine, and thyroxine.

Examples of the cationic species include a potassium ion, a sodium ion, a lithium ion, a calcium ion, a magnesium ion, and a zinc ion.

Specific examples of the active ingredient include a sodium salt of an amino acid, sodium chloride, potassium chloride, magnesium chloride, sodium citrate, magnesium citrate, sodium lactate, magnesium lactate, calcium lactate, sodium succinate, magnesium succinate, sodium malate, magnesium malate, sodium pyrrolidone carboxylate, magnesium pyrrolidone carboxylate, zinc sulfite, potassium aluminum sulfate (alum), seawater, and hot spring water.

Further, even in the case of the active ingredient that does not cause the movement of the magnesium ions and the hydroxide ions, by containing the sodium salt of the amino acid, the sodium chloride, the potassium chloride, the magnesium chloride, the sodium citrate, the magnesium citrate, the sodium lactate, the magnesium lactate, the calcium lactate, the sodium succinate, the magnesium succinate, the sodium malate, the magnesium malate, the sodium pyrrolidone carboxylate, the magnesium pyrrolidone carboxylate, the zinc sulfite, the potassium aluminum sulfate (the alum), the seawater, and the hot spring water described above, the magnesium ions and the hydroxide ions may be moved.

As described above, by a method of separately containing the sodium salt of the amino acid, the sodium chloride, the potassium chloride, the magnesium chloride, the sodium citrate, the magnesium citrate, the sodium lactate, the magnesium lactate, the calcium lactate, the sodium succinate, the magnesium succinate, the sodium malate, the magnesium malate, the sodium pyrrolidone carboxylate, the magnesium pyrrolidone carboxylate, the zinc sulfite, the potassium aluminum sulfate (the alum), the sea water, and the hot spring water, it is possible to use almost all generally commercially available pharmaceuticals, quasi-pharmaceuticals, cosmetics, and supplements as the active ingredient.

Examples of the pharmaceuticals, the quasi-pharmaceuticals, the cosmetics, and the supplements include the followings.

Examples of the pharmaceuticals include a live vaccine, an inactivated vaccine, a toxoid, a mRNA vaccine, a DNA vaccine, and as a viral vector vaccine, a Measles vaccine, a rubella vaccine, a Measles and rubella vaccine, a varicella vaccine, a mumps vaccine, a yellow fever vaccine, a BCG vaccine, a rotavirus vaccine, a pertussis vaccine, a Japanese encephalitis vaccine, an influenza vaccine, a hepatitis A vaccine, a hepatitis B vaccine, a Haemophilus influenzae type b (hib) vaccine, a 13-valent conjugate pneumococcal vaccine, a 23-valent capsular polysaccharide pneumococcal vaccine, a human papillomavirus vaccine, a rabies vaccine, an inactivated polio vaccine, a meningococcal vaccine, a diphtheria-tetanus vaccine, a DPT-IPV vaccine, a diphtheria vaccine for adults, a tetanus vaccine, a COVID-19 vaccine, and a SARS-COV-2 virus vaccine.

In addition, it is possible to use almost all commercially available pharmaceuticals and quasi-pharmaceuticals such as antasthmatics, insulin, growth hormones, peptides, an anticancer agent, traditional Chinese medicine, analgesics, an anti-inflammatory agent, nasal spray, and eye drops.

Examples of a material having an anti-aging effect include a uric acid, glutathione, melatonin, polyphenol, melanoidin, astaxanthin, kinetin, epigallocatechin gallate, coenzyme Q10, vitamins, superoxide dismutase, mannitol, quercetin, catechin and a derivative thereof, rutin and a derivative thereof, a moutan bark extract, an ivy extract, a Melissa extract, a Luohanguo extract, dibutyl hydroxy toluene, and butyl hydroxy anisole.

Examples of a material having a skin-lightening effect include a skin-lightening agent and an anti-inflammatory agent. The skin-lightening agent has an effect of preventing the occurrence of skin darkening caused by sunburn and the occurrence of blemishes and freckles caused by pigmentation. Examples of the skin-lightening agent include arbutin, an ellagic acid, a linoleic acid, vitamin C and a derivative thereof, a kojic acid, a tranexamic acid, a placenta extract, a chamomile extract, a licorice extract, a rose fruit extract, a scutellaria baicalensis extract, a seaweed extract, a sophora root extract, a milettia reticulata extract, an eleutherococcus extract, a rice bran extract, a wheat germ extract, an asiasarum root extract, a hawthorn extract, a chamaecrista mimosoides extract, a white lily extract, a peony extract, an inula flower extract, a soybean extract, a tea extract, a molasses extract, a Japanese ampelopsis root extract, a grape extract, a hop extract, a rose flower extract, a chaenomeles speciosa extract, and a saxifrage extract. The anti-inflammatory agent has an effect of suppressing inflammation of hot flashes and red spots on the skin after sunburn. Examples of the anti-inflammatory agent include sulfur and a derivative thereof, a glycyrrhizic acid and a derivative thereof, a glycyrrhetinic acid and a derivative thereof, an althea extract, an angelica keiskei extract, a chamomile extract, a lonicera flower extract, a watercress extract, a comfrey extract, a salvia extract, a lithospermi radix extract, a perilla extract, a birch extract, and a gentian extract.

Examples of a material having a peeling and brightening effect include an α-hydroxy acids, a salicylic acid, a sulfur, and urea.

Examples of a material having a slimming effect include a substance having an effect of promoting blood circulation, for example, a plant extract such as ginger, a capsicum tincture, and a Sophora flavescens root, carbon dioxide, and vitamin E and a derivative thereof.

Examples of a material having a moisturizing effect include a protein such as elastin and keratin, and a derivative thereof, hydrolysis, and a salt thereof, an amino acid such as glycine, serine, an aspartic acid, a glutamic acid, arginine, and theanine, and a derivative thereof, sorbitol, erythritol, trehalose, inositol, glucose, sucrose, and a derivative thereof, dextrin and a derivative thereof, saccharides such as honey, D-panthenol and a derivative thereof, sodium lactate, sodium pyrrolidone carboxylate, sodium hyaluronate, mucopolysaccharide, urea, a phospholipid, ceramide, a coptis japonica rhizome extract, an acorus calamus extract, a rehmannia root extract, an angelica polymorpha extract, a malva sylvestris extract, a horse chestnut extract, and a cydonia oblonga extract.

Examples of a material having a hair restoration effect include isopropyl methyl phenol, a Ginkgo biloba extract, L-menthol, carpronium chloride, diphenhydramine hydrochloride, polygonum (polygonum multiflorum), (dipotassium) glycyrrhizate, a salicylic acid, a dialkyl monoamine derivative, ginger, cepharanthine, powdered cnidium rhizome, swertia japonica, panax japonicus rhizome, panax ginseng, a capsicum tincture, angelica acutiloba, trehalose, a nicotinic acid/nicotinic acid amide, vitamin E (tocopherol), hinokitiol, a placenta extract, and pentadecanoic acid glyceride.

Examples of a material having a skin conditioning effect include a substance for the purpose of barrier function improvement or skin problem improvement such as a damage treatment. Examples of a material having a skin conditioning effect include ceramides, cholesterols, an amine derivative, caffeines, a cockscomb extract, a seashell extract, royal jelly, a silk protein, and a decomposition product and a derivative thereof, lactoferrin and a decomposition product thereof, mucopolysaccharides such as chondroitin sulfate and a hyaluronic acid, and a salt thereof, collagen, a yeast extract, a lactic acid bacterium extract, a bifidobacterium extract, a fermented metabolic extract, a Ginkgo biloba extract, a barley extract, a swertia japonica extract, a ziziphus jujuba fruit extract, a ginseng extract, an arnica extract, a termeric extract, a eucalyptus extract, a cattail extract, a Saponaria officinalis extract, a rosemary extract, a glycol extract, a citric acid, a lactic acid, a malic acid, a tartaric acid, and a succinic acid.

Examples of a material having a relaxing effect include lavender, rosemary, santalum album, orris, bitter orange, cypress, and orange oil.

Note that, only one type of such drug may be used alone, or two or more types thereof may be used in combination.

Examples of a cosmetic material include a toner, a milky lotion, a serum, a cream, a cream pack, a massage cream, a cleansing cream, a cleansing gel, a facial foam, a sunscreen, a styling gel, a shampoo, a body shampoo, a hair setting gel, a fragrance, and a hair dye. According to such cosmetic materials, effects of anti-aging, skin-lightening, peeling and brightening, slimming, moisturizing, hair restoration, hair growth, skin conditioning, relaxation, and ultraviolet protection can be obtained.

Note that, only one type of such cosmetic material may be used alone, or two or more types thereof may be used in combination.

### (Method for Producing Positive Electrode)

Next, a method for producing the positive electrode will be described.

First, a method for producing the bacteria-produced cellulose carbide configuring the positive electrode 201 will be described.

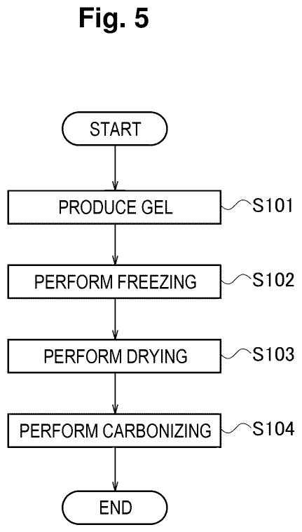

FIG. 5 is a flowchart illustrating the method for producing the bacteria-produced cellulose carbide.

In a gel producing step of step S101, a gel in which cellulose nanofibers are dispersed in predetermined bacteria is produced. In a freezing step of step S102, the gel produced by bacteria is frozen to be a frozen product. In a drying step of step S103, the frozen product is dried in vacuum. According to the above steps, a bacteria-produced xerogel is obtained. In a carbonizing step of step S104, the bacteria-produced xerogel is heated and carbonized in a gas atmosphere in which cellulose is not burned. Accordingly, the bacteria-produced cellulose carbide is obtained.

A gel means a solid in which a dispersion medium has lost fluidity due to a three-dimensional network structure of nanostructures serving as a dispersoid. Specifically, a gel means a dispersion system having a shear elastic modulus of 102 to 106 Pa. As the dispersion medium of the gel, an aqueous dispersion medium such as water (H₂O) can be used. Alternatively, as the dispersion medium of the gel, an organic dispersion medium such as a carboxylic acid, methanol (CH₃OH), ethanol (C₂H₅OH), propanol (C₃H₇OH), n-butanol, isobutanol, n-butylamine, dodecane, an unsaturated fatty acid, ethylene glycol, heptane, hexadecane, isoamyl alcohol, octanol, isopropanol, acetone, and glycerin can be used. Two or more types thereof may be mixed.

The gel produced by bacteria has a nanofiber (a fibrous substance having a diameter of 1 nm to 1 μm and a length of 100 times or more the diameter) of nm order as a basic structure. The positive electrode 201 produced by using such a gel has a high specific surface area. Since it is desirable that the positive electrode 201 of the patch 1 for sticking to a biological tissue has a high specific surface area, it is preferable to use the gel produced by bacteria. Specifically, by using the gel produced by bacteria, it is possible to synthesize the positive electrode 201 having a specific surface area of 300 m²/g or more.

The bacteria-produced gel has a structure in which fibers are entangled in a coil shape and a network shape, and further has a structure in which nanofibers formed by the growth of bacteria are branched. Therefore, the positive electrode 201 produced from the bacteria-produced gel attains excellent stretchability in which the strain at the elastic limit is 50% or more. Accordingly, the positive electrode 201 produced by using the bacteria-produced gel is capable of enhancing adhesion to the biological tissue.

Examples of the bacteria include known bacteria, and examples thereof include acetic acid bacteria such as Acetobacter xylinum subspecies sucrose fermentor, Acetobacter xylinum ATCC 23768, Acetobacter xylinum ATCC 23769, Acetobacter pasteurianus ATCC 10245, Acetobacter xylinum ATCC 14851, Acetobacter xylinum ATCC 11142, and Acetobacter xylinum ATCC 10821. Alternatively, the bacteria may be bacteria produced by culturing various mutant strains created by mutating the above-mentioned bacteria by a known method using nitrosoguanidine (NTG) or the like.

In the freezing step, for example, the bacteria-produced gel is contained in a suitable container such as a test tube, and the periphery of the test tube is cooled in a coolant such as liquid nitrogen to freeze the bacteria-produced gel. A method for freezing the bacteria-produced gel is not particularly limited insofar as the dispersion medium of the gel can be cooled to a freezing point or lower, and cooling may be performed in a freezer or the like. By freezing the bacteria-produced gel, the dispersion medium loses fluidity, cellulose serving as the dispersoid is fixed, and the three-dimensional network structure is constructed. In a case where the cellulose serving as the dispersoid is not fixed by freezing, in the subsequent drying step, the dispersoid is aggregated in accordance with the evaporation of the dispersion medium. Therefore, a sufficient high specific surface area is not obtained, and it is difficult to produce the positive electrode 201 with high performance.

The drying step is a step of drying a frozen product obtained in the freezing step to extract t the cellulose serving as the dispersoid maintaining or constructing the three-dimensional network structure from the dispersion medium. In the drying step, the frozen product is dried in vacuum, and the frozen dispersion medium is sublimated from a solid state. The drying step is performed, for example, by storing the obtained frozen product in an appropriate container such as a flask and vacuuming the inside of the container. As the frozen product is disposed in a vacuum atmosphere, the sublimation point of the dispersion medium drops, and a substance that is not sublimated at ordinary pressure can be sublimated. The degree of vacuum in the drying step varies with the dispersion medium being used, but is not limited to any specific degree as long as the dispersion medium sublimates. For example, in a case where water is used as the dispersion medium, the degree of vacuum needs to be set so that the pressure becomes 0.06 MPa or lower. However, heat is taken away as latent heat of sublimation, and therefore, drying takes time. In view of this, the degree of vacuum is preferably 1.0×10⁻⁶ to 1.0×10⁻² Pa. Furthermore, heat may be applied from a heater or the like during the drying. In a drying method in the atmosphere, the dispersion medium changes from a solid to a liquid and from a liquid to a gas. When the dispersion medium is in a liquid state, the dispersoid becomes fluid again in the dispersion medium, and the three-dimensional network structure of the cellulose collapses. therefore, it is difficult to produce the bacteria-produced cellulose carbide having stretchability by drying in an atmosphere at an atmospheric pressure.

The cellulose, which is a component contained in the bacteria-produced gel, does not have conductivity, and thus, it is important to perform a carbonizing step in which the cellulose is carbonized by a heat treatment in an inert gas atmosphere to impart conductivity. The bacteria-produced cellulose carbide is a three-dimensional network structure having conductivity. The bacteria-produced cellulose carbide has high conductivity, corrosion resistance, high stretchability, and a high specific surface area, and is preferable as the positive electrode 201 of the patch 1 for sticking to a biological tissue.

In the carbonizing step, the bacteria-produced xerogel may be carbonized by calcining the bacteria-produced xerogel at 500° C. to 2000° C., more preferably at 900° C. to 1800° C. in an inert gas atmosphere. Examples of the gas in which the cellulose is not burned include an inert gas such as nitrogen gas or argon gas. The gas to be used may be a reducing gas such as a hydrogen gas or a carbon monoxide gas, or may be a carbon dioxide gas. The carbon dioxide gas or the carbon monoxide gas that has an activating effect on a carbon material and can be expected to have high activation is more preferable.

Subsequently, a step of supporting the catalyst on the bacteria-produced cellulose carbide will be described.

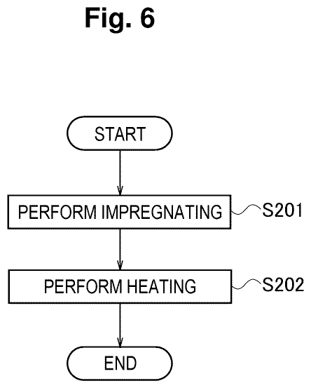

FIG. 6 is a flowchart illustrating the step of supporting the catalyst on the bacteria-produced cellulose carbide.

In an impregnating step of step S201, the bacteria-produced cellulose carbide obtained by the above-described production method is impregnated with an aqueous solution of a metal salt to be the precursor of the catalyst. In a heating step of step S202, the bacteria-produced cellulose carbide containing the metal salt is subjected to a heating treatment.

A preferred metal as the metal salt is at least one metal selected from the group consisting of iron, manganese, copper, nickel, silver, gold, platinum, cobalt, ruthenium, molybdenum, titanium, chromium, gallium, praseodymium, aluminum, silicon, and tin. Iron is preferable because of a low environmental load and high electrode performance.

In order to support a transition metal oxide on the bacteria-produced cellulose carbide, a known method of the related art can be used. For example, there are a method in which the bacteria-produced cellulose carbide is impregnated with an aqueous solution of a transition metal chloride or a transition metal nitrate, and evaporated to dryness, and then, hydrothermally synthesized in water at a high temperature and a high pressure, a precipitation method in which the bacteria-produced cellulose carbide is impregnated with an aqueous solution of a transition metal chloride or a transition metal nitrate, and an alkali aqueous solution is dropped thereto, and a sol-gel method in which the bacteria-produced cellulose carbide is impregnated with a transition metal alkoxide solution and hydrolyzed. The conditions for each of these methods according to liquid phase methods are known, and these known conditions can be applied. Since the transition metal oxide can be supported with high dispersion, such liquid phase methods are desirable.

In many cases, a metal oxide supported by the above liquid phase methods is in an amorphous state, because crystallization thereof has not progressed. The precursor in an amorphous state is subjected to a heat treatment in an inert atmosphere at a high temperature of approximately 500° C., so that a crystalline metal oxide can be obtained. Such a crystalline metal oxide exhibits high performance even in a case where it is used as the catalyst for the positive electrode.

On the other hand, a precursor powder obtained in a case where the amorphous precursor described above is dried at a relatively low temperature of approximately 100° C. to 200° C. is in the form of a hydrate while maintaining an amorphous state. The hydrate of the metal oxide can be formally expressed as MeₓO_{y}·nH₂O (where Me means the above metal, x and y each represents the number of metal and oxygen contained in the metal oxide molecule, and n represents the number of moles of H₂O with respect to 1 mole of metal oxide). The hydrate of the metal oxide obtained through such low-temperature drying can be used as the catalyst.

Having been hardly sintered, the amorphous metal oxide (the hydrate) has a large surface area, and a very small particle size of approximately 30 nm. This is suitable as a catalyst, and the use of this catalyst will lead to excellent battery performance.

As described above, the crystalline metal oxide exhibits high activity, but the surface area of the metal oxide crystallized through a high-temperature heat treatment as described above might decrease significantly. For example, the particle size may be approximately 100 nm due to the aggregation of the particles. Note that, this particle size (the average particle size) is the value obtained by enlarging and observing particles with a scanning electron microscope (SEM) or the like, measuring the diameter of the particles per 10 μm square (10 μm×10 μm), and calculating the average value.

In addition, particularly in the case of a catalyst based on a metal oxide subjected to a heat treatment at a high temperature, particles aggregate, and thus, it may be difficult to add the catalyst to the surface of the bacteria-produced cellulose carbide with high dispersion. To achieve a sufficient catalytic effect, it is necessary to add a large amount of metal oxide to the positive electrode in some cases, and the production of the catalyst through the heat treatment at a high temperature might be disadvantageous in terms of cost. In order to solve this problem, as described above, the amorphous precursor described above may be dried at a relatively low temperature of approximately 100° C. to 200° C.

The catalyst-unsupported bacteria-produced cellulose carbide or the catalyst-supported bacteria-produced cellulose carbide obtained by the above production method is processed into a plate-shaped product or a sheet, and the plate-shaped product or the sheet of the bacteria-produced cellulose carbide is cut into a desired rectangular shape (for example, 30 mm×20 mm) with a punching blade, a laser cutter, or the like to obtain the positive electrode 201.

Subsequently, another method for producing the positive electrode will be described.

The bacteria-produced cellulose carbide obtained by the above production method is brittle, and it may be difficult to process the bacteria-produced cellulose carbide into a desired shape. Therefore, it is easy to process the bacteria-produced cellulose carbide into a sheet shape by using another production method described below.

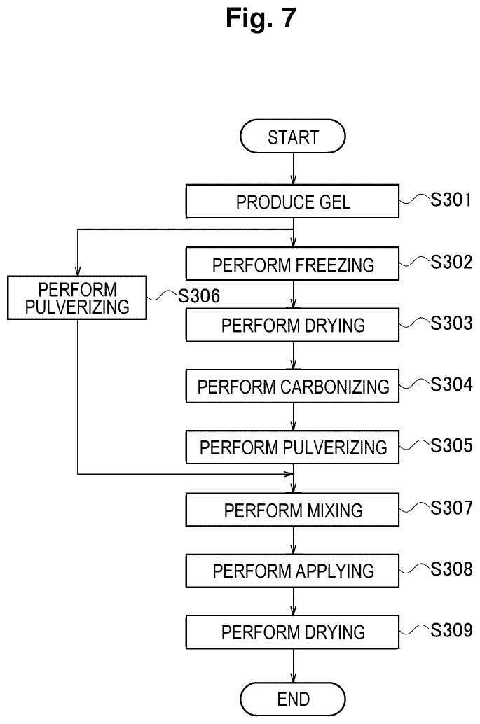

FIG. 7 is a flowchart illustrating another method for producing the positive electrode 201.

Steps S301 to S304 are similar to the method for producing the bacteria-produced cellulose carbide described in FIG. 5. After step S304, the step of supporting the catalyst on the bacteria-produced cellulose carbide described in FIG. 6 may be performed.

In a pulverizing step of step S305, the bacteria-produced cellulose carbide obtained in steps S301 to S304 is pulverized. In a pulverizing step of step S306, the bacteria-produced gel obtained in step S301 is pulverized. In a mixing step of step S307, the bacteria-produced cellulose carbide pulverized in step S305 and the bacteria-produced gel pulverized in step S306 are mixed.

In the pulverizing step, for example, the bacteria-produced gel and the bacteria-produced cellulose carbide are formed into a powder or slurry by using a mixer, a homogenizer, an ultrasonic homogenizer, a high-speed rotating shear type stirrer, a colloid mill, a roll mill, a high-pressure injection type disperser, a rotating ball mill, a vibrating ball mill, a planetary ball mill, or an attritor. In this case, the secondary particle size of the bacteria-produced gel and the bacteria-produced cellulose carbide is preferably 100 nm to 5 mm, and more preferably 1 μm to 1 mm. This is because when pulverization is performed until the secondary particle size is 100 nm or less, the co-continuous structure of the nanofibers breaks, it is difficult to obtain a sufficient binding force and a conductive path, and electrical resistance increases. In a case where the secondary particle size is 5 mm or more, the bacteria-produced gel functioning as a binding agent is not sufficiently dispersed, and it is difficult to maintain the positive electrode in a sheet shape.

Since the bacteria-produced cellulose carbide has a high porosity and a low density, in a case where the bacteria-produced cellulose carbide is pulverized alone, a powder of the bacteria-produced cellulose carbide flies during or after the pulverization, and thus, handling is difficult. Therefore, it is preferable that the bacteria-produced cellulose carbide is impregnated with a solvent, and then, pulverized. The solvent used here is not particularly limited, and for example, an aqueous solvent such as water (H₂O) can be used. Alternatively, as the solvent, an organic solvent such as a carboxylic acid, methanol (CH₃OH), ethanol (C₂H₅OH), propanol (C₃H₇OH), n-butanol, isobutanol, n-butylamine, dodecane, an unsaturated fatty acid, ethylene glycol, heptane, hexadecane, isoamyl alcohol, octanol, isopropanol, acetone, and glycerin can be used. Two or more types thereof may be mixed.

It is also possible to simultaneously pulverize the bacteria-produced gel and the bacteria-produced cellulose carbide. In this case, the mixing step can be omitted, which is preferable.

A mixture prepared by the pulverizing step and the mixing step is in the shape of slurry. In an applying step of step S308, the mixed slurry is applied to a part of the conductive layer 203. In a drying step of step S309, the applied mixed slurry is dried.

According to the above steps, the sheet-shaped positive electrode 201 can be processed into a desired shape.

In the applying step, the mixed slurry may be applied to either the soluble microneedles 3 and 301A or the conductive layer 203, and in the case of applying the mixed slurry to the soluble microneedles 3 and 301A, the soluble microneedles absorb and dissolve the solvent at the time of application, and thus, it is preferable to apply the mixed slurry to the conductive layer 203.

In the drying step, a thermostatic bath, a vacuum dryer, an infrared dryer, a hot air dryer, or a suction dryer may be used. By performing suction filtration using an aspirator or the like, rapid drying can be performed.

As another method, the mixed slurry may be dried into the shape of a sheet, and then, processed into a desired shape. For example, the obtained sheet-shaped bacteria-produced cellulose carbide is cut into a desired rectangular shape (for example, 30 mm×20 mm) with a punching blade, a laser cutter, or the like to obtain the positive electrode 201. However, the material cost of the scraps and the like generated by a cutting process increases, compared to the method of applying the mixed slurry.

The positive electrode 201 may be produced by using cellulose nanofiber carbon instead of the bacteria-produced cellulose carbide. The production method using the cellulose nanofiber carbon is the same as the production method using the bacteria-produced cellulose carbide.

Specifically, as in the production method of FIG. 5, in the freezing step, a solution containing cellulose nanofibers is frozen to obtain a frozen product. In the drying step, the frozen product is dried in vacuum to obtain a dried product. In a carbonizing step, the dried product is carbonized by heating in a gas atmosphere in which cellulose is not burned. Accordingly, the cellulose nanofiber carbon is obtained. The cellulose nanofiber carbon produced by this production method has a fibrous network structure. This cellulose nanofiber carbon has a three-dimensional network structure having conductivity, and has physical property values, characteristics, and performance equivalent to those of bacteria-produced cellulose carbide. The cellulose nanofiber carbon is processed into a plate shape or a sheet, and cut into a desired shape to obtain the positive electrode 201. Note that, as in the step of FIG. 6, a catalyst may be supported on the cellulose nanofiber carbon.

In addition, as in the production method of FIG. 7, slurry may be produced from the cellulose nanofiber carbon, and the slurry may be applied and dried to produce the positive electrode 201. In a pulverizing step, the cellulose nanofiber carbon prepared as described above is pulverized. In a mixing step, a cellulose nanofiber solution and the pulverized cellulose nanofiber carbon are mixed. Accordingly, a slurry-shaped mixture is obtained. In an applying step and a drying step, the mixed slurry is applied to the conductive layer 203 and dried.

### (Method for Producing Negative Electrode)

Next, a method for producing the negative electrode will be described.

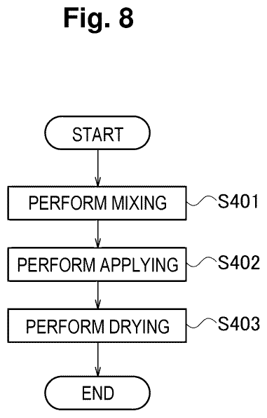

FIG. 8 is a flowchart illustrating the method for producing the negative electrode 202.

In a mixing step of step S401, a metal powder containing predetermined magnesium, a binder, and a conductive aid are mixed. In an applying step of step S402, the mixed slurry obtained by mixing is applied to a part of the conductive layer 203. In a drying step of step S403, the applied mixed slurry is dried. According to the above steps, the negative electrode 202 can be produced. In the manufacturing method of FIG. 8, the material cost can be suppressed, and the thin and flexible negative electrode 202 can be produced, compared to the method for cutting out a magnesium foil into a predetermined shape.

In the mixing step, for example, a magnetic stirrer, a stirrer, a mixer, a rotating and revolving mixer, a vacuum stirring and defoaming mixer, a mixer, a homogenizer, an ultrasonic homogenizer, a high-speed rotating shear type stirrer, a colloid mill, a roll mill, a high-pressure injection type disperser, a rotating ball mill, a vibration ball mill, a planetary ball mill, or an attritor is used to prepare the slurry containing the metal powder containing magnesium, the binder, and the conductive aid.

The metal powder containing magnesium to be mixed can be pure magnesium and an alloy mainly containing magnesium. Examples of the alloy mainly containing magnesium include AZ31, AZ31B, AZ61, AZ91, AMX601, AMX602, AZX611, AZX612, AM50, AM60, and LZ91.

For the synthesis of the metal powder containing magnesium, it is possible to use a method for synthesizing a magnesium powder of the related art. Examples thereof include a water atomization method, a gas atomization method, a centrifugal atomization method, a melt spinning method, a rotary electrode method, a stamp/mill method, a ball mill method, a mechanical alloying method, an oxide reduction method, a chloride reduction method, a hydrometallurgical method, an electrolytic carbonyl reaction method, and a hydrogen plasma irradiation method.

The particle size of the metal powder containing magnesium may be 10 nm to 5 μm, and preferably 20 nm to 2 μm. This is because in a case where the particles are excessively large, it is difficult to make contact between the particles when coating and drying are performed, and the electrical conductivity is lowered. In a case where the particles are excessively fine, an oxidation reaction may proceed and magnesium may be inactivated. In some cases, there is a concern that the magnesium metal is burned due to rapid progress of the oxidation reaction, which leads to a fire accident.

The binder to be mixed may be a binder that binds the particles to each other after the drying step of the slurry. The binder may be gum arabic, sodium alginate, curdlan, carrageenan, agar, xanthan gum, chitosan, guar gum, a konjac powder, cyclodextrin, gelatin, tamarind gum, tara gum, dextrin, starch, pregelatinized starch, pullulan, pectin, egg albumen, locust bean gum, propylene glycol, glycerin, soybean albumin, CMC, cellulose, or bacteria-produced cellulose, which does not contain fluorine and is used as a food additive. The pulverized bacteria-produced cellulose used for the production of the positive electrode 201 is preferable as the binder because a structure in which nanofibers are three-dimensionally entangled firmly binds the metal powder containing magnesium. Since the bacteria-produced cellulose is a material required for synthesizing the positive electrode 201, the same material can be used for the positive electrode 201 and the negative electrode 202, which is advantageous in terms of cost.

The conductive aid to be mixed, for example, may be bacteria-produced cellulose carbide, a carbon powder, or a conductive polymer, and a conductive polymer having a high binding property with the metal powder containing magnesium is preferable. Examples of the conductive polymer include polyacetylene, which is an aliphatic conjugated system, poly (p-phenylene), which is an aromatic conjugated system, poly (p-phenylene vinylene) and polythienylene vinylene, which are a mixed conjugated system, polypyrrole, polythiophene, and polyethylene dioxythiophene (PEDOT), which are a heterocyclic conjugated system, polyaniline, which is a hetero atom-containing conjugated system, polyacene and polyfluorene, which are a multi-chain conjugated system, and graphene, which is a two-dimensional conjugated system. PEDOT, which has excellent conductivity and excellent environmental stability in a conductor state, is preferable.

In the mixing step, a solvent may be added in addition to the metal powder containing magnesium, the binder, and the conductive aid. The solvent is not particularly limited, and for example, an aqueous solvent such as water (H₂O) can be used. Alternatively, as the solvent, an organic solvent such as a carboxylic acid, methanol (CH₃OH), ethanol (C₂H₅OH), propanol (C₃H₇OH), n-butanol, isobutanol, n-butyl amine, dodecane, an unsaturated fatty acid, ethylene glycol, heptane, hexadecane, isoamyl alcohol, octanol, isopropanol, acetone, and glycerin can be used. Two or more types thereof may be mixed.

In the applying step, the mixed slurry may be applied to either the soluble microneedles 3 and 301B or the conductive layer 203, and as with the positive electrode 201, it is preferable to apply the mixed slurry to the conductive layer 203.

In a case where both of the slurry for a positive electrode and the slurry for a negative electrode are applied to the conductive layer 203, the drying step may be performed after both of the slurry for a positive electrode and the slurry for a negative electrode are applied to the conductive layer 203.

In addition to the above production method, the negative electrode 202 can be formed by a known method. For example, the negative electrode 202 is produced by molding a metallic magnesium foil into a predetermined shape.

### EXAMPLES AND EVALUATION RESULTS

Next, a plurality of examples of a patch for sticking to a biological tissue and evaluation results thereof will be described.

### Example 1

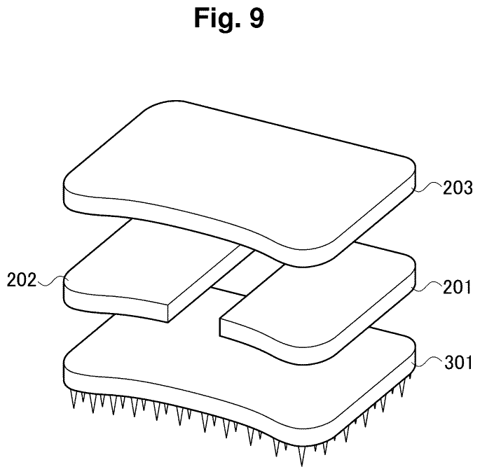

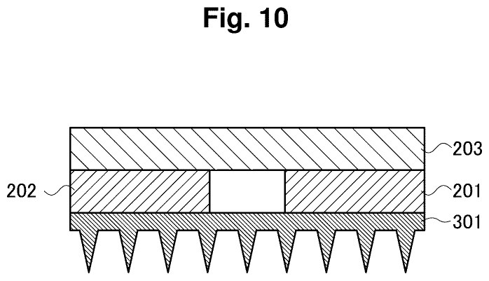

FIG. 9 is an exploded perspective view of a patch for sticking to a biological tissue according to Example 1, and FIG. 10 is a sectional view of the patch for sticking to a biological tissue according to Example 1.

The patch for sticking to a biological tissue of Example 1 includes the positive electrode 201, the negative electrode 202, a soluble microneedle 301, and the conductive layer 203. In Example 1, bacteria-produced cellulose carbide was used for the positive electrode 201. Hereinafter, the preparation of the patch for sticking to a biological tissue of Example 1 will be described.

The bacteria-produced cellulose carbide used for the positive electrode 201 was obtained by the following method.

A bacteria cellulose gel produced by Acetobacter xylinum as acetic acid bacteria was used as a bacteria-produced gel, and the bacteria-produced gel was immersed in liquid nitrogen for 30 minutes in an expanded polystyrene box to be completely frozen. After completely freezing the bacteria-produced gel, the frozen bacteria-produced gel was taken out on a petri dish and dried in vacuum of 10 Pa or less by a freeze dryer (manufactured by TOKYO RIKAKIKAI CO., LTD.) to obtain a bacteria-produced xerogel. After drying in vacuum, the bacteria-produced xerogel was carbonized by calcination at 1200° C. for 2 hours in a nitrogen atmosphere to obtain bacteria-produced cellulose carbide.

The obtained bacteria-produced cellulose carbide was evaluated by performing XRD measurement, SEM observation, porosity measurement, a tensile test, and BET specific surface area measurement. This bacteria-produced cellulose carbide was confirmed to be a carbon (C, PDF card No. 01-071-4630) single phase by XRD measurement. The PDF Card No. is a card number in Powder Diffraction File (PDF), which is a database constructed by the International Centre for Diffraction Data (ICDD). It was confirmed by SEM observation that the bacteria-produced cellulose carbide was a co-continuous body in which nanofibers having a diameter of 20 nm were continuously connected. The BET specific surface area of the bacteria-produced cellulose carbide was measured with a BET apparatus and found to be 830 m²/g. The porosity of the bacteria-produced cellulose carbide was measured by a mercury intrusion technique, and was found to be 99% or more. The porosity was calculated by modeling fine pores as cylindrical shapes from a pore size distribution obtained by calculating the bacteria-produced cellulose carbide by the mercury intrusion technique. From the results of the tensile test, it was confirmed that the elastic region was not exceeded and the shape was restored to the shape before stress application even when 80% of strain was applied by tensile stress, and it was found that excellent stretchability was exhibited even after carbonization.

The positive electrode 201 was prepared by cutting out the obtained bacteria-produced cellulose carbide into a rectangular shape of 30 mm×20 mm with a punching blade, a laser cutter, or the like.

The negative electrode 202 was prepared by cutting out a commercially available metallic magnesium foil (a thickness of 200 μm, manufactured by The Nilaco Corporation) into a rectangular shape of 30 mm×20 mm with a punching blade, a laser cutter, or the like.

In order to prepare the soluble microneedle 301, molds corresponding to 200 microneedles per 1 square cm were prepared by laser processing. By using this mold, a soluble microneedle sheet having a needle length of 0.4 mm, containing sodium hyaluronate and a human insulin protein, was prepared. Specifically, a mixture in which water, the sodium hyaluronate, and the human insulin protein were adjusted at a weight ratio of 90:9:1 was poured into the mold, dried in a dryer at 60° C. for 24 hours, and then, peeled off from the mold to prepare a soluble microneedle sheet. The soluble microneedle sheet was cut into a rectangular shape of 30 mm×50 mm with a punching blade to prepare the soluble microneedle 301. Here, the sodium hyaluronate was used as a base material of the soluble microneedle 301, and the human insulin protein was used as an active ingredient.

The conductive layer 203 was prepared by cutting out a commercially available carbon cloth (manufactured by Toray Industries, Inc.) into a rectangular shape of 30 mm×50 mm with a punching blade.

By using the above constituents, the patch for sticking to a biological tissue was prepared as follows. First, the positive electrode 201 and the negative electrode 202 are stacked on the conductive layer 203, and the positive electrode 201 and the negative electrode 202 are interposed between the conductive layer 203 and the soluble microneedle 301. At this time, the positive electrode 201 and the negative electrode 202 are disposed on the conductive layer 203 with a space therebetween such that the positive electrode 201 and the negative electrode 202 are not in contact with each other. Subsequently, 1 mm inside of the outer periphery of each of the positive electrode 201 and the negative electrode 202 was sewn and press-bonded by using a sewing machine to obtain a patch for sticking to a biological tissue.

In order to confirm the storage performance of the patch for sticking to a biological tissue, the patch for sticking to a biological tissue was stored for 1 week in a dark room in which the room temperature was maintained at 25° C. before the patch for sticking to a biological tissue was inserted into a biological tissue, and then, the patch for sticking to a biological tissue was inserted into the biological tissue to start a battery reaction.

First, in order to start the battery reaction, the soluble microneedle 301 of the patch for sticking to a biological tissue was disposed such that the surface of the soluble microneedle was in contact with the biological tissue, and a pressure was applied with a finger to firmly insert the soluble microneedle 301 into the biological tissue. When the soluble microneedle 301 is inserted into the biological tissue, the base material of the soluble microneedle 301 and the active ingredient are dissolved in the biological tissue by the moisture in the biological tissue to function as an electrolytic solution, and the battery reaction is started.

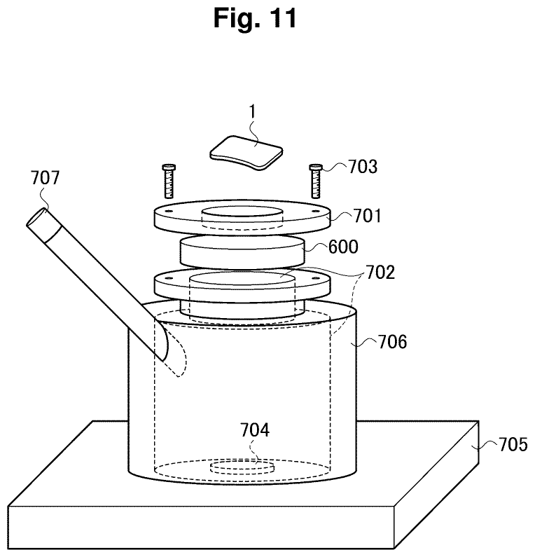

In an evaluation test, the patch for sticking to a biological tissue was disposed on a test device illustrated in FIG. 11, and the skin permeability of the active ingredient (the human insulin protein) to a test piece (the human excised skin) was confirmed.

The test device illustrated in FIG. 11 includes a donor portion 701 and a receiver portion 702. A test piece 600 is used by being interposed between the donor portion 701 and the receiver portion 702 and fixed with a stopper 703. The material of the donor portion 701 and the receiver portion 702 can be plastic, a metal, glass, ceramic, or the like. Here, Teflon (Registered Trademark) was used for the donor portion 701, and glass was used for the receiver portion 702. The receiver portion 702 was filled with an aqueous solution having a pH adjusted to 7.4 with a phosphate buffer solution from a sampling port 707. Constant temperature water of 35° C. was circulated in a jacket portion 706 of the test device. A stirrer 704 was put in the receiver portion 702, and stirring was continuously and slowly performed by using a magnetic stirrer 705.

The test piece 600 is obtained by using the human excised skin having a thickness of 700 μm and hydrating the skin with a phosphate buffer solution having a pH of 7.4 for 30 minutes. When the test piece 600 (the human excised skin) was fixed to the test device, the test piece was disposed such that the horny cell layer side was on the donor portion 701 side and the dermis side was on the receiver portion 702 side.

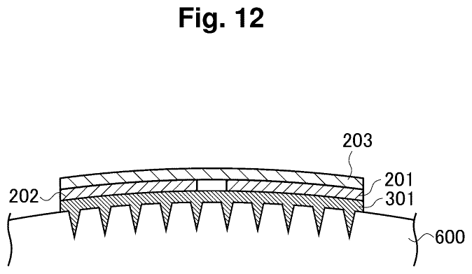

As illustrated in FIG. 12, the test piece 600 to which the patch for sticking to a biological tissue that has started the battery reaction is attached is provided in contact with the phosphate buffer solution filled in the lower portion of the receiver portion 702. The active ingredient oozes out into the phosphate buffer solution through the test piece 600. The solution was taken out from the donor portion 701 at regular time intervals, and the cumulative permeation amount with respect to the test piece 600 was calculated.

The concentration was measured by high performance liquid chromatography (manufactured by Agilent Technologies Japan, Ltd.). As a column, Agilent Poroshell 120 EC-C18 of 4.6×100 mm was used. As a mobile phase, a solution prepared by adjusting 20 mmol of dihydrogen phosphate buffer (KH₂PO₄) to pH 2.5 with an o-phosphoric acid, and 60% methanol/40% acetonitrile were used. The flow rate was measured at 1.5 mL/min.

Note that, measurement results of Example 1 will be described below together with measurement results of Comparative Example 1 described below.

### Comparative Example 1

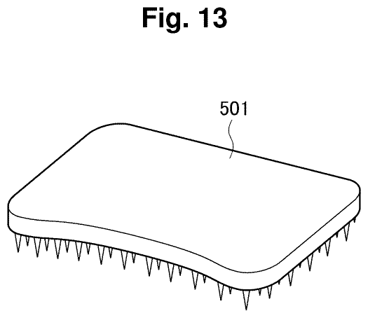

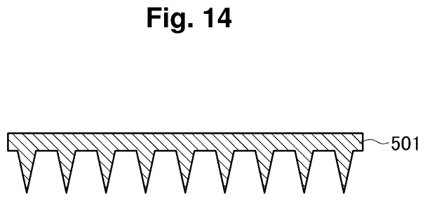

FIG. 13 is a perspective view of a patch for sticking to a biological tissue according to Comparative Example 1, and FIG. 14 is a sectional view of the patch for sticking to a biological tissue according to Comparative Example 1.

As a comparative example not including a battery unit, a patch 501 for sticking to a biological tissue using only the same soluble microneedle and the same active ingredient as those in Example 1 was prepared.

As with Example 1, the patch 501 for sticking to a biological tissue was prepared by cutting out a soluble microneedle sheet containing sodium hyaluronate and a human insulin protein into a rectangular shape of 30 mm×50 mm with a punching blade. That is, the patch 501 for sticking to a biological tissue of Comparative Example 1 is the same as the soluble microneedle 301 of Example 1.

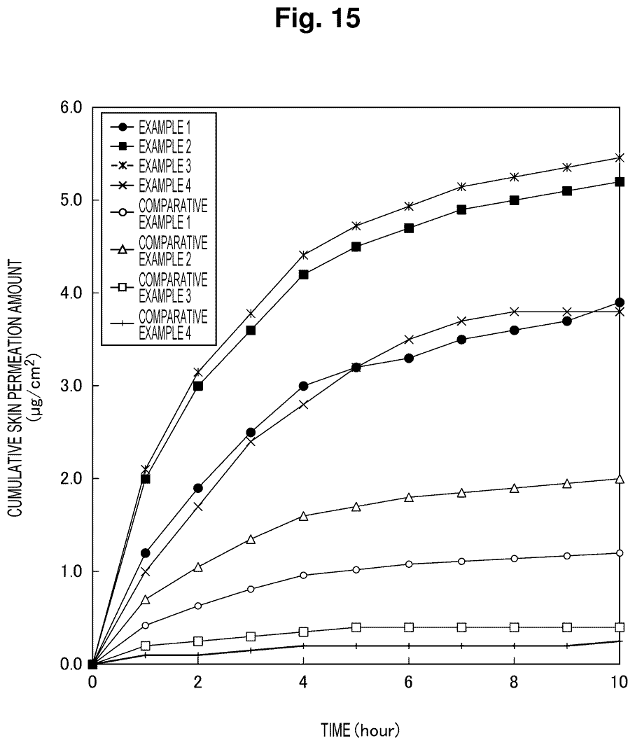

FIG. 15 illustrates the measurement results of Example 1 and Comparative Example 1. Note that, FIG. 15 also illustrates measurement results of Examples 2 to 4 and Comparative Examples 2 and 3 described below.

As is clear from the measurement results illustrated in FIG. 15, in Example 1, the cumulative permeation amount of the human insulin protein increased with the lapse of time. This is considered to be because the human insulin protein was also introduced into the biological tissue at the same time as the movement of hydroxide ions into the biological tissue accompanying the battery reaction.

In contrast, in Comparative Example 1, there was no significant change in the cumulative permeation amount of the human insulin protein.

Hereinafter, Examples 2 to 4 and Comparative Examples 2 to 4 will be described in order.

### Example 2

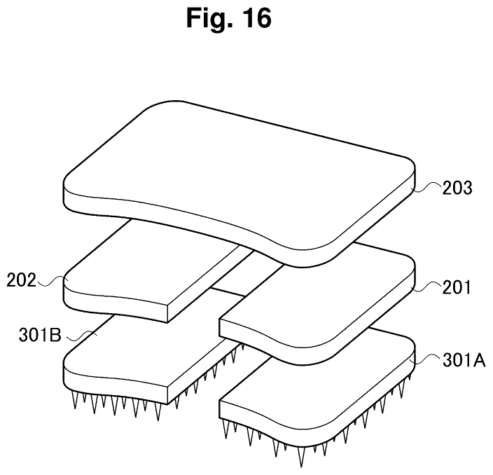

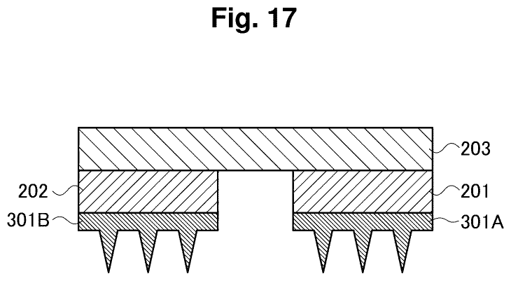

FIG. 16 is an exploded perspective view of a patch for sticking to a biological tissue of Example 2, and FIG. 17 is a sectional view of the patch for sticking to a biological tissue of Example 2.

Example 2 is different from Example 1 in that the positive electrode part soluble microneedle 301A and the negative electrode part soluble microneedle 301B are provided apart from each other. Two soluble microneedle sheets of Example 1 were cut into a rectangular shape of 30 mm×20 mm with a punching blade to obtain the positive electrode part soluble microneedle 301A and the negative electrode part soluble microneedle 301B.

A preparation method, a test device, and an evaluation method of the patch for sticking to a biological tissue are the same as those in Example 1.

From the measurement results shown in FIG. 15, it is found that in Example 2, the cumulative permeation amount of the human insulin protein at each time increases, compared to Example 1 and Comparative Example 1. In Example 1, the ions were moved through not only the biological tissue but also the soluble microneedle 301, and the effect of ion introduction was suppressed. In Example 2, by dividing the soluble microneedle into the positive electrode part soluble microneedle 301A and the negative electrode part soluble microneedle 301B, the movement of the ions through the biological tissue was promoted, and the effect of ion introduction was increased.

### Example 3

The configuration of a patch for sticking to a biological tissue of Example 3 is the same as that of Example 2.

Example 3 is different from Example 2 in that the positive electrode 201 and the negative electrode 202 are applied to the conductive layer 203 by using the production methods in FIGS. 7 and 8.

First, a method for producing the positive electrode 201 of Example 3 will be described. As with Example 1, a bacteria-produced gel and bacteria-produced cellulose carbide are produced. In a pulverizing step and a mixing step, the bacteria-produced cellulose carbide was impregnated with water, and then, the bacteria-produced gel and the bacteria-produced cellulose carbide were stirred at a weight ratio of 1:1 with a homogenizer (manufactured by SMT Co., Ltd.) for 12 hours. In an applying step, slurry for a positive electrode obtained in the mixing step was applied to the conductive layer 203 with a thickness of 3 mm and a width of 30 mm×20 mm using a squeegee.

A method for producing the negative electrode 202 of Example 3 will be described. Flame-retardant magnesium AZX612 (manufactured by Gonda Metal Industry Co., Ltd.) containing 1 wt % of zinc, 2 wt % of calcium, and 6 wt % of aluminum in magnesium was used for the negative electrode 202. Nanoparticles of the flame-retardant magnesium AZX612 were synthesized by irradiating the flame-retardant magnesium AZX612 with hydrogen plasma using a metal nanoparticle production device (manufactured by ATTOTEC Co., Ltd.). As a result of SEM observation of the nanoparticles, the average particle size was approximately 100 nm, and from the results of ICP optical emission spectrometry, d that no compositional deviation occurred even when granularization was performed.

The bacteria-produced gel was used as a binder for the negative electrode 202. A bacteria-produced gel is prepared as with Example 1. The bacteria-produced gel was stirred in a homogenizer (manufactured by SMT Co., Ltd.) for 12 hours to obtain a slurry-shaped bacteria-produced gel.

As a conductive aid of the negative electrode 202, an aqueous dispersion liquid (5.0 wt %, Orgacon EL-P-5015, manufactured by Sigma-Aldrich Co. LLC.) containing a mixture of polyethylene dioxythiophene and polyanion poly(styrene sulfonate) was used.

In a mixing step, a metal powder containing magnesium, the slurry-shaped bacteria-produced gel, and the conductive aid were stirred for 24 hours by using a ball mill to obtain slurry for a negative electrode.

In an applying step, the slurry for a negative electrode obtained in the mixing step was applied to the conductive layer 203 after applying the slurry for a positive electrode with a thickness of 3 mm and a width of 30 mm×20 mm using a squeegee. The slurry for a negative electrode is applied onto the conductive layer 203 separately from the slurry for a positive electrode.

The conductive layer 203 coated with the slurry for a positive electrode and the slurry for a negative electrode was dried at 60° C. for 24 hours by using a thermostatic bath to obtain the positive electrode 201 and the negative electrode 202.

As with Example 2, the positive electrode part soluble microneedle 301A and the negative electrode part soluble microneedle 301B were prepared, and press-bonded by a sewing machine to prepare a patch for sticking to a biological tissue.

From the measurement results illustrated in FIG. 15, it is found that in Example 3, the cumulative permeation amount of the human insulin protein at each time increases, compared to Examples 1 and 2 and Comparative Example 1. In Example 3, since the positive electrode 201 and the negative electrode 202 were applied to the conductive layer 203, an adhesive force with the conductive layer 203 was strong, a resistance value was reduced, and the ion introduction by the battery reaction was promoted.

### Example 4

The configuration of a patch for sticking to a biological tissue of Example 4 is the same as that of Example 1. Example 4 is different from Example 1 in that cellulose nanofiber carbon is used for the positive electrode 201 instead of the bacteria-produced cellulose carbide.

The cellulose nanofiber carbon used for the positive electrode 201 was obtained by the following method.

First, 1 g of cellulose nanofibers and 10 g of ultrapure water were stirred with a homogenizer (manufactured by SMT Co., Ltd.) for 12 hours by using cellulose nanofibers (manufactured by Nippon Paper Industries Co., Ltd.) to obtain a cellulose nanofiber solution in which the cellulose nanofibers were dispersed.

A test tube containing the cellulose nanofiber solution was immersed in liquid nitrogen for 30 minutes to completely freeze the cellulose nanofiber solution. The frozen cellulose nanofiber solution was taken out on a petri dish and dried in vacuum of 10 Pa or less by a freeze dryer (manufactured by TOKYO RIKAKIKAI CO., LTD.) to obtain a dried product of the cellulose nanofiber. After drying in vacuum, the cellulose nanofibers were carbonized by calcination at 600° C. for 2 hours in a nitrogen atmosphere to obtain cellulose nanofiber carbon.

This cellulose nanofiber carbon was confirmed to be a carbon (C, PDF card No. 01-071-4630) single phase by XRD measurement. It was confirmed by SEM observation that the cellulose nanofiber carbon was a co-continuous body in which nanofibers having a diameter of 70 nm were continuously connected. The BET specific surface area of the cellulose nanofiber carbon was measured with a BET apparatus and found to be 690 m²/g. The porosity of the cellulose nanofiber carbon was measured by a mercury intrusion technique, and was found to be 99% or more. From the results of a tensile test, it was confirmed that the elastic region was not exceeded and the shape was restored to the shape before stress application even when 30% of strain was applied by tensile stress, and it was found that excellent stretchability was exhibited even after carbonization.

A preparation method, a test device, and an evaluation method of the patch for sticking to a biological tissue are the same as those in Example 1.

From the measurement results illustrated in FIG. 15, it is found that in Example 4, the cumulative permeation amount of the human insulin protein at each time increases, compared to Comparative Example 1. In addition, it is found that the cumulative permeation amount of the human insulin protein at each time is comparable with that in Example 1. This is because the cellulose nanofiber carbon used for the positive electrode 201 has an excellent specific surface area as with the bacteria-produced cellulose carbide, and by the fibrous network structure of the cellulose nanofiber carbon, a battery overvoltage is suppressed and ion introduction is promoted.

### Comparative Example 2

The configuration of a patch for sticking to a biological tissue of Comparative Example 2 is the same as that of Example 1. Comparative Example 2 is different from Example 1 in that carbon (Ketjen Black EC600JD) known as electrodes for air electrodes of general magnesium-air batteries was used for a positive electrode.

Specifically, a Ketjen black powder (manufactured by Lion Corporation), and a polytetrafluoroethylene (PTFE) powder (manufactured by Daikin Industries, Ltd.) were sufficiently pulverized and mixed at a weight ratio of 50:30:20 with a mortar machine, and were subjected to roll molding to form a sheet-shaped electrode with a thickness of 0.5 mm. The sheet-shaped electrode was cut into a size of 30 mm×20 mm to obtain a positive electrode of Comparative Example 2.

A preparation method, a test device, and an evaluation method of the patch for sticking to a biological tissue are the same as those in Example 1.

From the measurement results illustrated in FIG. 15, in Comparative Example 2, the cumulative permeation amount of the human insulin protein at each time was smaller than that in Examples 1 to 4. In addition, when the positive electrode of Comparative Example 2 was observed after measurement, a part of the positive electrode was broken, and contamination due to the carbon powder was confirmed in the biological tissue.

### Comparative Example 3

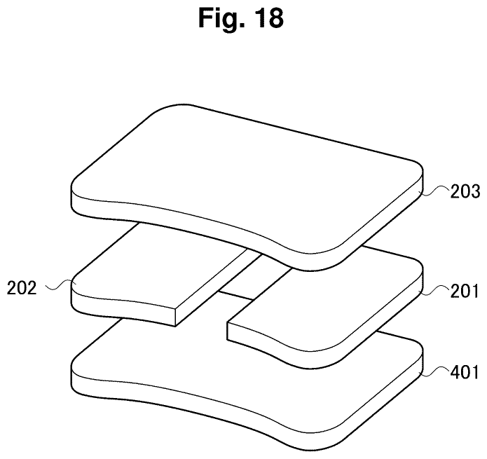

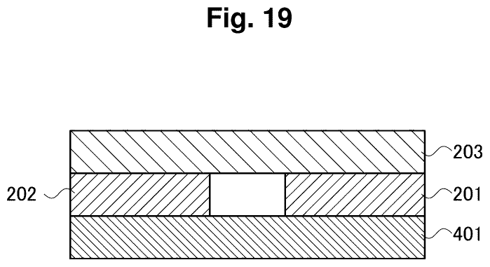

FIG. 18 is an exploded perspective view of a patch for sticking to a biological tissue according to Comparative Example 3, and FIG. 19 is a sectional view of the patch for sticking to a biological tissue according to Comparative Example 3.

In Comparative Example 3, the soluble microneedle of Example 1 was replaced with a cotton 401, and the cotton 401 was immersed in the active ingredient. Specifically, in Comparative Example 3, the positive electrode 201, the negative electrode 202, and the conductive layer 203 were produced as with Example 1. In Comparative Example 3, instead of the soluble microneedle, a commercially available cellulosic cotton (Bemcot, manufactured by Asahi Kasei Corporation) was cut into a rectangular shape of 30 mm×50 mm with a punching blade to produce the cotton 401. The cotton 401 was directly immersed in a human insulin protein as the active ingredient.

A test device and an evaluation method are the same as those in Example 1.

From the measurement results illustrated in FIG. 15, in Comparative Example 3, the cumulative permeation amount of the human insulin protein at each time was smaller than that in Examples 1 to 4 and Comparative Examples 1 and 2. This is considered to be because in Comparative Example 3, the soluble microneedle was not used, and a high-molecular-weight drug (the human insulin protein) having a molecular weight of 500 daltons or more was not capable of permeating the skin barrier. The human insulin protein has a molecular weight of 5808 daltons.

### Comparative Example 4

In Comparative Example 4, the patch for sticking to a biological tissue of Comparative Example 3 was stored in a state where the cotton 401 was immersed in the active ingredient (that is, in a state where the positive electrode 201 and the negative electrode 202 were in contact with the active ingredient).

Specifically, in Comparative Example 4, a patch for sticking to a biological tissue was prepared as with Comparative Example 3, and then, sufficiently impregnated the cotton 401 with the active ingredient (the human insulin protein), and then, stored in a dark room maintained at a room temperature of 25° C. for 1 week. After that, the stored patch for sticking to a biological tissue was taken out and evaluated as with Example 1.

From the measurement results illustrated in FIG. 15, in Comparative Example 4, the cumulative permeation amount of the human insulin protein at each time was smaller than that in Examples 1 to 4 and Comparative Examples 1 to 3. In Comparative Example 4, since the active ingredient was stored in contact with the positive electrode 201 and the negative electrode 202, deterioration due to self-discharge of the battery, the corrosion of the negative electrode, the alteration of the active ingredient, and the like occurred.

As described above, the patch 1 for sticking to a biological tissue of this embodiment is the patch for sticking to a biological tissue that is used by being stuck to the biological tissue, the patch for sticking to a biological tissue, including: the battery unit 2; and the soluble microneedle 3 in contact with the battery unit 2, in which the soluble microneedle 3 contains the active ingredient, and the battery reaction is started by inserting the soluble microneedle 3 into the biological tissue 100.

In this embodiment, by using the soluble microneedle 3, it is possible to obtain an excellent ion introduction effect in which the permeation of a high-molecular-weight active ingredient having a molecular weight of 500 daltons or more is available. In addition, it is possible to promote the permeation of the high-molecular-weight active ingredient. In addition, in this embodiment, by inserting the soluble microneedle 3 into the biological tissue 100, it is possible to easily start the battery reaction. A user can easily use the patch 1 for sticking to a biological tissue.

In addition, the battery unit of the patch 1 for sticking to a biological tissue of this embodiment does not include the electrolyte, and the soluble microneedle 3 is dissolved in the biological tissue 100, and thus, the soluble microneedle 3 functions as the electrolyte, and the battery reaction is started. As a result, in this embodiment, it is possible to provide the patch 1 for sticking to a biological tissue that suppresses the self-discharge of the battery unit 2 during storage and can be stored for a long period of time.

According to this embodiment, by using the bacteria-produced cellulose carbide or the cellulose nanofiber carbon for the positive electrode 201 of the battery unit 2, it is possible to reduce the environmental load and easily perform the disposal in daily life. In addition, the three-dimensional network structure of the bacteria-produced cellulose carbide or the cellulose nanofiber carbon is capable of suppressing the battery overvoltage and promoting the ion introduction.

The present invention is not limited to the above-described embodiment, and it is apparent that various modifications and combinations can be implemented by those skilled in the art without departing from the technical spirit of the present invention.

### REFERENCE SIGNS LIST

1 PATCH FOR STICKING TO BIOLOGICAL TISSUE
2 Battery unit
201 Positive electrode
202 Negative electrode
203 Conductive layer
3, 301 Soluble microneedle
301A Positive electrode part soluble microneedle
301B Negative electrode part soluble microneedle
401 Cotton
100 Biological tissue

1. A patch for sticking to a biological tissue that is used by being stuck to a biological tissue, the patch comprising:
a battery unit; and
a soluble microneedle in contact with the battery unit,
wherein the soluble microneedle contains an active ingredient, and
a battery reaction is started by inserting the soluble microneedle into the biological tissue.

2. The patch for sticking to a biological tissue according to claim 1,
wherein the soluble microneedle functions as an electrolyte and the battery reaction is started by dissolving the soluble microneedle in the biological tissue.

3. The patch for sticking to a biological tissue according to claim 1,
wherein the battery unit includes a positive electrode, a negative electrode, and a conductive layer, and
the conductive layer is in contact with each of the positive electrode and the negative electrode.

4. The patch for sticking to a biological tissue according to claim 3,
wherein the soluble microneedle includes a positive electrode part soluble microneedle that is disposed to be in contact with the positive electrode and not to be in contact with the negative electrode, and a negative electrode part soluble microneedle that is disposed to be in contact with the negative electrode and not to be in contact with the positive electrode, and
the positive electrode part soluble microneedle and the negative electrode part soluble microneedle are used in a state of being inserted into the biological tissue.

5. The patch for sticking to a biological tissue according to claim 3,
wherein the positive electrode contains cellulose carbide having a three-dimensional network structure.

6. The patch for sticking to a biological tissue according to claim 3,
wherein the negative electrode contains at least one selected from the group consisting of magnesium, zinc, aluminum, iron, calcium, lithium, and sodium.

7. The patch for sticking to a biological tissue according to claim 2,
wherein the battery unit includes a positive electrode, a negative electrode, and a conductive layer, and
the conductive layer is in contact with each of the positive electrode and the negative electrode.
